(19) United States
(12) Patent Application Publication
(10) Pub. No.: US 2012/0258087 A1
(43) Pub. Date: Oct. 11, 2012

(54) ISOTONIC BEVERAGE WITH CHELATES

(75) Inventors: Marcin Jedlinski, Debica (PL); Rafal Jedlinski, Debica (PL); Piotr Kula, Debica (PL); Justyna Widlak-Kargul, Klimontow (PL); Gertruda Birus-Marszalek, Brzeznica (PL)

(73) Assignee: OLIMP LABORATORIES SP. Z O.O., Debica (PL)

(21) Appl. No.: 13/515,665

(22) PCT Filed: Jan. 4, 2010
(86) PCT No.: PCT/PL10/00001

§ 371 Date: Jun. 13, 2012

(30) Foreign Application Priority Data
Dec. 14, 2009 (PL) P-389921

(51) Int. Cl. A23L 2/38; A61K 38/02; A61K 31/522; A61K 36/18; A61K 36/82; A61K 36/258; A61K 36/752; A61K 31/122; A61P 7/00; A61P 19/02; A23L 1/304; A23L 2/44; A23L 2/60; A23L 1/303; A23L 2/39; A61K 31/198

(52) U.S. Cl. 424/94.1; 514/565; 514/1.1; 514/263.34; 424/725; 424/729; 424/728; 424/736; 514/675; 426/541; 426/73

(57) ABSTRACT

The subject of the invention is an isotonic beverage with chelates containing carbohydrates, mineral amino acid chelates, L-carnitine, sweeteners, flavouring substances, and a diluent, the essential feature of the beverage being the content of up to 0.1 wt % of mineral components in the form of mineral amino acid chelates, preferably bisglycinates and glycinates, from 0.1 to 50 wt % of isomaltulose, up to 15 wt % of L-carnitine and/or its derivatives, from 5 to 84% of carbohydrates, up to 50 wt % of bioactive substances, up to 50 wt % of flavouring substances, up to 80 wt % of polyalcohols, diluents, tableting aids, stabilizers, antioxidants, dyes, and 0.013-25 wt % of sweeteners, whereby its osmolarity is 275-295 mOsm/kg and pH is 2-5.

The subject of the invention is an isotonic beverage with chelates in the form of a liquid, semi-liquid suspension, or a solid improving water-electrolyte balance and allowing hydration of a human body, intended especially for active individuals and sportsmen, exerting strenuous physical effort.

Drinking suitable drinks by sportsmen before, during, and after a strenuous physical effort, especially during training, is an important issue since even little physical effort is accompanied with a loss of elements important for health such as potassium, sodium, calcium and chloride ions. Electrolytic balance disorders frequently lead to decreased resistance of a human body to physical effort. In order to solve problems related to the electrolytic balance of a human body it is necessary to drink suitable beverages with well-balanced content of electrolytes and osmotic pressure adjusted to the human body fluids. They can be avoided by immediate replenishment of these fluid thereby limiting the dehydration of human body.

The studies on the diet of different social groups carried out also by National Food and Nutrition Institute in Warsaw have revealed too low consumption of mineral nutrients: calcium, magnesium, potassium, zinc, and copper while the content of sodium and phosphorus in the diet was found to be too high. This is directly related to the high processing degree of food available on the market, which is rich in refined carbohydrates and saturated unhealthy fats. Moreover, low consumption of vegetables and fruits rich in microelements aggravates nutrient deficiencies. Without doubt dietary supplements are needed not only by sportsmen. The problem in their use is low absorption of the most popular inorganic mineral nutrients. Living organisms are not able to absorb inorganic minerals in their natural form. In order to utilize an inorganic microelement, a living organism must transform it into an organic biological form, which can be easily assimilated. Therefore, in human gastrointestinal tract a process has to take place of chemical binding of inorganic mineral molecules into organic compounds which can later be absorbed and utilized. This process is called chelation and its product is called a chelate. Unfortunately, the environment of the gastrointestinal tract is not an ideal place for efficient natural chelation process to occur. There are many factors which interfere with this process, therefore it is very inefficient. First, very often there is no sufficient amount of proper organic nutrients, therefore the inorganic mineral is not absorbed. There are very many chemical compounds interfering with the natural chelation process, for example some minerals in inorganic form which mutually prevent their absorption. Moreover, minerals in inorganic form are very unstable chemically. In acidic pH of stomach and duodenum they dissociate into ions, which very often are bound into harmful compounds causing irritation of the upper gastrointestinal tract. The reaction of further segments of the gastrointestinal tract, where the actual absorption of all nutrients takes place, changes into basic (pH increases). In such an environment the inorganic minerals precipitate in the form of poorly soluble salts, which cannot be absorbed. This is often manifested as stomach pain and constipations like in the case of iron sulphate. The scientists for many years have been working on a method allowing increased efficiency of the absorption of mineral compounds from oral supplements.

A beverage for sportsmen was described in U.S. Pat. No. 0,587,972A1 containing minerals of sodium, potassium, calcium, and magnesium, wherein at least a half of sodium mineral is sodium citrate, sodium chloride or their mixture, at least of a half of magnesium mineral is carbonate salt soluble in the aqueous solution of citric acid and malic acid, and at least a half of potassium mineral is potassium citrate. Moreover, this beverage optionally contains a supplement of ascorbic acid and 1-14 wt % of fruit juice as a source of sugar. Apple and grape juices or their mixture are used as a fruit juice and pH of this beverage is from 2.0 to 5.0.

A formulation is described in U.S. Pat. No. 1,252,781 for limiting dehydration and fatigue during physical exercise, for the replacement of fluids, salts and minerals lost during intensive physical activity which contains from 16 to 26 meq Na⁺, from 1.5 to 4.7 meq K⁺, from 11.9 to 19.9 meq Cl⁻, from 4.1 to 10.1 meq PO₄³⁻ and from 30 to 60 g of glucose per liter of this beverage, or containing 0.85 g of NaCl, 0.15 g of NaH₂PO₄, 0.15 g of KH₂PO₄, 0.10 g of KCl, 0.15 g of NaHCO₃ and 5 g of glucose per liter of the beverage, while a solution based on these formulas contains also absorbed CO₂ and shows pH below 3.0.

An isotonic fruit beverage for children is described in Pat. No. US 2000/0132034A1 containing water, electrolytes, at least 7 wt % of a source of the carbohydrate, a source of calcium, Vitamin C, showing 300-380 Osm/kg and pH of 3.0-4.5, wherein the fruit juice constitutes from 8 to 12 wt %, total carbohydrate content is 5-7 wt %, electrolyte content is from 60 to 250 mg/l of sodium and about 60 to about 150 mg/l of potassium, pH is from 3.5 to 4.2, and osmolarity is from 340 to 370 Osm/kg.

A rehydration drink described in U.S. Pat. No. 5,397,786 is a beverage for the treatment and prevention of the loss of electrolytes important for a human body in the case of dehydration. According to this invention creatine, Vitamin B, panthotenic acid and choline together with the addition of numerous salt such as MgCO₃, CaCO₃, and magnesium aspartate as supplements containing important energetic components have a function of energy enhancers.

On the other hand the isotonic beverage, known under the trade name “Gatorade” preventing dehydration of the body during physical exercise taking place at high temperature and at high moisture in the air, is composed of a mixture of carbohydrates in the form of a 6% solution and electrolytes such as sodium and potassium. It contains 6 g of a protein, 52 mg of sodium, 47 mg of chlorides, 12 mg of potassium, and 5 mg of magnesium in 100 ml of this beverage.

The purpose of the invention is to develop such a qualitative and quantitative composition of an isotonic beverage with highly absorptive chelated minerals, which could allow significant improvement of the electrolyte balance of the human body and that would fulfil the fundamental requirement of normal interaction with human body, i.e. having the osmotic pressure of the body fluids from 275 to 295 mOsm/kg. Further goal of the invention is the development of such a formula of an isotonic beverage, which will be able to be produced both in the form of a liquid and a powdered concentrate soluble in water, which will play a function of both rehydrating beverage during increased physical effort and sport activity, and which will provide very effective replenishment of the lacking mineral nutrients lost during the exercise, allowing normal function of the body.

The isotonic beverage with chelates of the current invention contains more than 0.1 wt % of the mineral components in the form of the chelates of amino acid minerals, preferably bisglycinates and glycinates, from 0.1 to 50 wt % of isomaltulose, up to 15 wt % of L-carnitine and/or its derivatives, 5 to 84% of carbohydrates, up to 50 wt % of bioactive substances, up to 50 wt % of flavouring substances, up to 80 wt % of polyalcohols, diluents, tableting aids, stabilizers, antioxidants and dyes and 0.013-25% wt % of sweeteners, while its osmolarity is 275-295 mOsm/kg and pH is 2-5. As mineral components in the form of bisglycinates, the beverage contains magnesium and/or calcium and/or potassium glycinate, and preferably contains also glycinates of zinc, iron, manganese, molybdenum, chromium, copper and/or selenium.

Besides, as mineral components organic and/or inorganic substances are used or as mineral components preferably used are chelates of aminoacids other than glycine, preferably L-arginine, L-methionine, L-lysine, and L-asparagine acid. In this beverage glucose and/or fructose, sucrose or isomaltulose function as carbohydrates, whereas as bioactive substances used are magnesium creatine chelate, oligopeptides, preferably glutamine peptide, proteins, preferably whey protein isolate, caffeine, salt, taurine, theanine, unsaturated fatty acids, preferably omega 3-6-9 fatty acids, astaxanthin, ubiquinone, preferably coenzyme Q10, hydroxymethyl butyrate (HMB), vegetable and fruit extracts, preferably green tea extract, ginseng extract, citrus fruit extract or other active substances, preferably chemical compounds showing physiological action.

Moreover, as flavouring substances used are synthetic, synthetic-natural substances, or natural substances in the form of a liquid, powder, emulsion or seasoning, preferably grapefruit juice concentrate or cherry juice concentrate, orange juice concentrate, orange flavour, pineapple flavour, apple flavour, lemon flavour, pineapple-strawberry seasoning and forest fruit flavour, preferably raspberry-blackberry flavour, whereas as stabilizers used are Gum Arabic, guar, carrageen gum, cellulose gum, xanthan gum, glycerol esters and vegetable resin, and as antioxidants preferably citric acid or sodium citrate or calcium lactate or ascorbic acid or calcium ascorbate are used.

Additionally, the beverage contains dyes, preferably brilliant green and/or quinoline yellow or Allura Red, or brilliant blue, or ammonia caramel, or preservatives, preferably sodium benzoate and/or potassium sorbate, as well as sweeteners such as liquid glucose and crystalline sugar or sugar powder, or aspartame and/or acesulphame K and/or cyclamic acid and its salts and/or saccharin or its salts and/or sucralose. The isotonic beverage of the current invention contains also polyalcohols, preferably sorbitol, mannitol, xylitol, and erithritol, and diluents, preferably fructose, sorbitol, mannitol, xylitol or erithritol, as well as anti-caking agents, preferably magnesium stearate and colloidal silica and vitamins, preferably vitamin premix composed of Vitamin A, C, B₁, B₂, B₆, B₁₂, and H, panthotenic acid, Vitamin D and E, niacin, and folic acid. This beverage has the form of an instant powder for dissolution in water or a liquid solution.

The basic advantage of the isotonic beverage of the current invention is its versatile action depending on the formula applied, namely the beverage containing L-carnitine, Sinetrol, HMB, and green extract tea as basic components enhances weight loss, while that containing Q10 (ubiquinone), grape extract, L-carnitine, and astaxanthin enhances heart beat. Furthermore, the isotonic beverage containing green tea extract, grape extract, bisglycinates of zinc, manganese and copper as basic components of the formula may function as antioxidative beverage, that containing extracts of aloe and selenium glycinate enhances immunity, that containing magnesium creatine chelate, taurine and ginseng extract as basic components is an energy drink, while that containing hyaluronic acid and glucosamine sulphate as basic components reinforces joints in humans.

Besides, the application of mineral amino acid chelates being combination of two amino acids with a mineral molecule, characterized by high chemical stability, results in increased resistance to acidic and basic environment of the gastrointestinal tract, thereby preventing dissociation into ions analogically to inorganic forms of minerals, which in turn results in their high bioavailability and absorption. Moreover, they are resistant to the action of antinutritional components and do not inhibit the action of the vitamins contained. Application in the beverage of the current invention different forms of amino acid chelates allows faster and more effective replacement of lost electrolytes, increased rehydration and regular nutrition of muscles under great physical effort.

On the other hand, the application of isomaltulose and mineral amino acid chelates allowed development of an unknown before innovative isotonic beverage which enhances rehydration of human body and regular nutrition of muscles under physical effort.

The subject of the invention was shown in embodiments of its formulas for liquid and powder forms which do not limit its application and in the examples of the liquid form based on the instant powder isotonic beverage, and direct liquid form of this beverage.

The examples of the formulas of the liquid form and instant powder isotonic beverage (in g/1000 ml of the liquid or suspension) are shown in Tables 1-7.

TABLE 1

| Formula components for | | | |
|---|---|---|---|
| the liquid and powder | Embodiments of the formula composition | | |
| form of the isotonic | (in g/1000 ml of liquid or suspension) | | |
| beverage | I | II | III |
| glucose | 10.639 | 13.5 | 10 |
| fructose | 10.639 | 13.5 | 10 |
| crystalline sugar (sucrose) | 8.149 | 10.5 | 10 |
| maltodextrins | — | 15 | 10 |
| salt | 0.379 | 0.38 | 0.38 |
| sodium citrate | 1.397 | 1.398 | 1.398 |
| potassium glycinate | 1 | 1 | 1 |
| calcium bisglycinate | 0.27 | — | — |
| magnesium bisglycinate | 0.278 | 0.278 | 0.278 |
| L-carnitine tartrate | 0.318 | 0.296 | 0.296 |
| isomaltulose | — | — | 12.5 |
| glutamine peptide | 2.945 | — | — |
| grapefruit juice concentrate | 11.589 | — | — |
| masking flavour | 0.192 | — | — |
| grapefruit flavour | 0.603 | — | — |
| blue flavour seasoning | — | 0.64 | 0.64 |
| forest fruit flavour | — | 0.36 | 0.36 |
| malic acid | 3.6 | 3.6 | 3.6 |
| potassium sorbate | 0.25 | 0.25 | 0.25 |
| sodium benzoate | 0.075 | 0.075 | 0.075 |
| acesulphame K | 0.134 | — | — |
| mixture of sweeteners | — | 0.07 | — |
| containing aspartame, | | | |
| acesulphame K, sodium | | | |
| sucrate, sodium cyclamate | | | |
| thickener (Gum Arabic) | — | — | — |
| quinoline yellow (E104) | 0.003 | — | — |
| brilliant blue (E133) | — | 0.003 | — |
| brilliant green (E142) | 0.003 | — | — |
| water | 947.54 | 939.153 | 939.223 |
| Total: | 1000 | 1000 | 1000 |

TABLE 2

| | Embodiments of the formula | | |
|---|---|---|---|
| Formula components for the | composition (in g/1000 ml | | |
| liquid and powder form of | of liquid or suspension) | | |
| the isotonic beverage | IV | V | VI |
| glucose | 13.5 | 13.5 | 13.0 |
| fructose | 13.5 | 10 | 14.3 |
| crystalline sugar (sucrose) | 10.5 | 10.5 | 11.2 |
| maltodextrins | 14.82 | 14 | 16 |
| salt | 0.38 | 0.38 | 0.38 |
| sodium citrate | 1.398 | 1.398 | 2.091 |
| potassium glycinate | 1 | 1 | 1 |
| magnesium bisglycinate | 0.278 | — | — |
| dipotassium hydrogen | — | — | 0.26 |
| phosphate | | | |
| magnesium hydroxycarbonate | — | — | 0.2 |
| ascorbic acid | | | 0.12 |
| L-camitine tartrate | 0.296 | 0.296 | 0.296 |
| isomaltulose | — | 3.5 | — |
| hyaluronic acid | 0.01 | — | — |
| glucosamine sulphate 2KCl | 0.154 | — | — |
| magnesium bisglycinate | 0.00625 | — | — |
| magnesium creatine chelate | — | 1 | — |
| taurine | — | 1 | 3.869 |
| caffeine | — | — | 0.21 |
| glucuronolactone | — | — | 2.519 |
| pineapple-strawberry | — | — | 16.741 |
| seasoning | | | |
| blue flavour seasoning | 0.64 | 0.64 | — |
| forest fruit flavour | 0.36 | 0.36 | — |
| citric acid | — | — | 3.396 |
| malic acid | 3.6 | 3.6 | — |
| potassium sorbate | 0.25 | 0.25 | 0.3 |
| sodium benzoate | 0.075 | 0.075 | 0.075 |
| mixture of sweeteners | — | — | 0.05 |
| (aspartame, acesulphame K, | | | |
| sodium sucrate, sodium | | | |
| cyclamate) | | | |
| brilliant green (E142) | — | — | 1.5 |
| water | 939.24 | 938.5 | 912.523 |
| Total: | 1000 | 1000 | 1000 |

TABLE 3

| | Embodiments of the formula | | |
|---|---|---|---|
| Formula components for the | composition (in g/1000 ml | | |
| liquid and powder form of | of liquid or suspension) | | |
| the isotonic beverage | VII | VIII | IX |
| glucose | 13.5 | 15.498 | 10 |
| fructose | 13.5 | 9.551 | 10 |
| crystalline sugar (sucrose) | 10.5 | — | 10 |
| maltodextrins | 15 | 19.679 | 10 |
| sugar powder | — | 38.717 | — |
| salt | 0.38 | 1.182 | 0.38 |
| sodium citrate | 1.398 | 0.314 | 1.398 |
| potassium glycinate | 1 | 2.7 | 1 |
| calcium bisglycinate | — | 0.55 | — |
| magnesium bisglycinate | 0.278 | 0.4 | — |
| magnesium hydroxycarbonate | — | — | 0.1984 |
| vitamin premix | — | 0.246 | — |
| L-carnitine tartrate | 0.296 | 0.42 | 0.296 |
| isomaltulose | — | 0.7 | 7.5 |
| magnesium bisglycinate | 0.03 | — | — |
| magnesium creatine chelate | — | 0.2 | — |
| glutamine peptide | — | 3.152 | — |
| copper bisglycinate | 0.02 | — | — |
| selenium glycinate | 0.01 | — | 0.01 |
| grape rind extract | — | — | 0.1 |
| green tea extract | — | — | 0.25 |
| forest flavour | — | — | 0.3 |
| blue flavour seasoning | 0.64 | — | — |
| forest fruit flavour | 0.36 | — | — |
| grapefruit flavour | — | 0.936 | — |
| orange flavour | — | 0.844 | — |
| malic acid | 3.6 | 4.033 | 3.6 |
| potassium sorbate | 0.25 | — | 0.25 |
| sodium benzoate | 0.075 | — | 0.075 |
| magnesium stearate | — | 0.308 | — |
| sucralose | — | 0.0209 | — |
| acesulphame K | — | 0.0449 | — |
| sorbitol | — | 0.1 | — |
| mannitol | — | 0.1 | — |
| erithritol | — | 0.1 | — |
| xylitol | — | 0.1 | — |
| E110 dye | — | 0.0043 | — |
| colloidal silica | — | 0.1 | — |
| Starsweet mixture of sweeteners | — | — | 0.1 |
| (aspartame, acesulphame K, sodium | | | |
| sucrate, sodium cyclamate) | | | |
| thickener (Gum Arabic) | — | — | 0.2 |
| water | 939.163 | — | 944.343 |
| Total: | 1000 | 100 | 1000 |

TABLE 4

| Formula components for the | Embodiments of the formula composition | | |
|---|---|---|---|
| liquid and powder form of | (in g/1000 ml of liquid or suspension) | | |
| the isotonic beverage | X | XI | XII |
| glucose (meritose) | 11.5 | 13 | 19.878 |
| fructose | 11.5 | 13 | 19.878 |
| crystalline sugar | 8 | 10 | — |
| maltodextrins | 11.5 | 14 | 22.2878 |
| sugar powder | — | — | 14.457 |
| salt | 0.38 | 0.38 | 0.572 |
| sodium citrate | 1.398 | 1.398 | 2.1022 |
| potassium glycinate | 1 | 1 | 1.5059 |
| magnesium bisglycinate | 0.278 | 0.278 | — |
| magnesium | — | — | 0.301 |
| hydroxycarbonate | | | |
| L-carnitine tartrate | 0.296 | 0.42 | 0.4457 |
| isomaltulose | 4.5 | — | 11.294 |
| manganese bisglycinate | 0.03 | — | — |
| copper bisglycinate | 0.02 | — | — |
| selenium glycinate | 0.01 | 0.01 | 0.15059 |
| grape rind extract | — | — | 0.1506 |
| green tea extract | 0.3 | — | 0.379 |
| patented mixture of citrus | 0.5 | — | — |
| fruit extracts and guarana | | | |
| (Sinetrol) | | | |
| aloe extract | — | 0.5 | — |
| vitamin B6 | 0.0024 | 0.0024 | — |
| Niacin | 0.018 | 0.018 | — |
| calcium panthotenate | 0.006 | 0.006 | — |
| aloe flavour | — | 0.015 | — |
| aloe extract flavour | — | 1 | — |
| grapefruit flavour | 0.3 | — | 0.7228 |
| malic acid | 3.2 | 2.6 | 5.4213 |
| potassium sorbate | 0.25 | 0.25 | — |
| sodium benzoate | 0.075 | 0.075 | — |
| Starsweet mixture of | 0.13 | 0.062 | 0.15059 |
| sweeteners (aspartame, | | | |
| acesulphame K, sodium | | | |
| sucrate, sodium cyclamate) | | | |
| thickener (Gum Arabic) | — | — | 0.3012 |
| Allura Red (E129) | — | — | 0.0043 |
| brilliant green (E142) | — | — | 0.003 |
| water | 936.196 | 943 | — |
| Total: | 1000 | 1000 | 100 |

TABLE 5

| Formula components for the | Embodiments of the formula composition | | |
|---|---|---|---|
| liquid and powder form of | (in g/1000 ml of liquid or suspension) | | |
| the isotonic beverage | XIII | XIV | XV |
| Glucose (meritose) | 20.9426 | 28.1139 | 23.504 |
| fructose | 20.9426 | 21.498 | 20.645 |
| maltodextrins | 20.9426 | 23.153 | 22.233 |
| sugar powder | 16.1097 | 15.876 | 15.246 |
| salt | 0.612 | 0.628 | 0.603 |
| sodium citrate | 2.2489 | 2.308 | 2.217 |
| potassium glycinate | 1.611 | 1.653 | 1.588 |
| magnesium bisglycinate | 0.4446 | 0.456 | 0.438 |
| L-carnitine tartrate | 0.4768 | 0.486 | 0.470 |
| isomaltulose | 7.249 | — | 11.9108 |
| selenium glycinate | — | 0.0165 | — |
| Green tea extract | 0.6444 | — | — |
| Patented mixture of citrus | 1.611 | — | — |
| fruit extracts and guarana | | | |
| (Sinetrol) | | | |
| aloe extract | — | 0.826 | — |
| Vitamin B6 | 0.0038 | 0.00396 | — |
| niacin | 0.0289 | 0.0297 | — |
| calcium panthotenate | 0.00966 | 0.0099 | — |
| sweetness enhancing flavour | — | — | 0.318 |
| lemon flavour | 0.7732 | — | 0.826 |
| apple flavour | — | 0.4625 | — |
| malic acid | 5.155 | 4.299 | — |
| brilliant blue (E133) | — | — | 0.019 |
| Total: | 100 | 100 | 100 |

TABLE 6

| Formula components for the | Embodiments of the formula composition | | |
|---|---|---|---|
| liquid and powder form of | (in g/1000 ml of liquid or suspension) | | |
| the isotonic beverage | XVI | XVII | XVIII |
| glucose | 13.5 | 13.5 | 13.5 |
| fructose | 13.5 | 13.5 | 13.5 |
| crystalline sugar (sucrose) | 10.5 | 10.5 | 10.5 |
| maltodextrins | 15 | 15 | 15 |
| salt | 0.38 | 0.38 | 0.38 |
| sodium citrate | 1.398 | 1.398 | 1.398 |
| potassium glycinate | 1 | 1 | 1 |
| magnesium bisglycinate | 0.278 | — | — |
| iron bisglycinate | 0.07 | — | — |
| magnesium bisglycinate | — | 0.025 | 0.025 |
| zinc methionate | — | | 0.0375 |
| zinc arginate | — | 0.15 | — |
| calcium ascorbate | 0.15 | — | — |
| Vitamin B6 | — | 0.004 | 0.004 |
| L-carnitine tartrate | — | 0.296 | 0.296 |
| Omega 3 | 0.2 | — | — |
| Q10 coenzyme | 0.03 | — | — |
| astaxanthin | 0.133 | — | — |
| blue flavour seasoning | 0.64 | 0.64 | 0.64 |
| forest fruit flavour | — | 0.36 | 0.36 |
| pineapple flavour | 0.36 | — | — |
| malic acid | 3.6 | 3.6 | 3.6 |
| potassium sorbate | 0.25 | 0.25 | 0.25 |
| sodium benzoate | 0.075 | 0.075 | 0.075 |
| glycerol and vegetable resin | 0.01 | — | — |
| esters | | | |
| Gum arabic | 0.01 | — | — |
| carrageen gum | 0.01 | — | — |
| cellulose gum | 0.01 | — | — |
| xanthane gum | 0.01 | — | — |
| Starsweet mixture of | — | 0.07 | 0.07 |
| sweeteners (aspartame, | | | |
| acesulphame K, sodium | | | |
| sucrate, sodium cyclamate) | | | |
| water | 938.886 | 939.252 | 939.364 |
| Total: | 1000 | 1000 | 1000 |

TABLE 7

| Formula components for the | Embodiments of the formula composition | | |
|---|---|---|---|
| liquid and powder form of | (in g/1000 ml of liquid or suspension) | | |
| the isotonic beverage | XIX | XX | XXI |
| glucose | 13.5 | 13.5 | 13.5 |
| fructose | 13.5 | 13.5 | 13.5 |
| crystalline sugar (sucrose) | 10.5 | 10.5 | 10.5 |
| maltodextrins | 15 | 15 | 15 |
| salt | 0.38 | 0.38 | 0.38 |
| sodium citrate | 1.398 | 1.398 | 1.398 |
| potassium glycinate | — | — | 1 |
| magnesium bisglycinate | — | 0.556 | 0.278 |
| zinc bislysinate | — | 0.0375 | — |
| magnesium asparaginate | 0.025 | — | — |
| zinc asparaginate | 0.0375 | — | — |
| Vitamin B6 | 0.004 | 0.004 | — |
| L-carnitine tartrate | — | 0.296 | 0.296 |
| whey protein isolate | — | — | 5 |
| calcium hydroxymethyl | — | — | 1 |
| butyrate | | | |
| Omega 3 | 0.2 | — | — |
| Q10 Coenzyme | 0.03 | — | — |
| astaxanthin | 0.133 | — | — |
| L-theanine | | 0.1 | |
| blue flavour seasoning | 0.64 | 0.64 | 0.64 |
| forest fruit flavour | 0.36 | 0.36 | 0.36 |
| malic acid | 3.6 | 3.6 | 3.6 |
| potassium sorbate | 0.25 | 0.25 | 0.25 |
| sodium benzoate | 0.075 | 0.075 | 0.075 |
| glycerol and vegetable resin | 0.01 | — | — |
| esters | | | |
| guar gum | 0.01 | — | — |
| carrageen gum | 0.01 | — | — |
| cellulose gum | 0.01 | — | — |
| xanthane gum | 0.01 | — | — |
| Starsweet mixture of | 0.07 | 0.07 | 0.07 |
| sweeteners (aspartame, | | | |
| acesulphame K, sodium | | | |
| sucrate, sodium cyclamate) | | | |
| water | 940.247 | 939.733 | 939.153 |
| Total: | 1000 | 1000 | 1000 |

### EMBODIMENT XXII

The components of the isotonic beverage are dosed according to one of the formulas given in Embodiments XIII-XX into a bin of suitable volume.

The next stage is mixing of the raw materials placed in the bin using properly programmed mixer in order to obtain homogeneous mixture. Intermediate product after thorough mixing and release after organoleptic tests by Quality Control is transferred for packing, and depending on the needs it is packed into HDPE containers of different volume or into multilayer foil sachets.

The product obtained this way in the instant form is intended for dissolving in water in a suitable proportion and after thorough mixing it is ready for use. Osmolarity of this drink, depending on the formula composition is 275-295 mOsm, and pH is 2-5.

### EMBODIMENT XXII

The initial stage of the production of the isotonic beverage, according to one of selected formulas given in Embodiments XIII-XX is dosing the components according to the approved formula, then these components are dissolved using a system of high volume mixers using technological water and water obtained using reverse osmosis.

The beverage obtained this way, after confirming compliance of physicochemical parameters, including required osmolarity of 275-295 mOsm, and acidity of pH=2-5, is released for the next stage of the manufacture by Quality Control.

Moreover, depending on the type of packaging, the beverage undergoes tunnel or flow pasteurization in order to ensure microbiological stability of the finished product, whereas the beverage is going to be poured into PET bottles and undergoes flow pasteurization at 110-115° C. for 120 s, and then is cooled with an ice water down to 7-9° C. and is passed for bottling.

Closed containers are labelled with the production batch number and the expiry date, and then labelled with sleeve-type labels.

The beverage is then poured into aluminum tins and after closing undergoes tunnel pasteurization at the temperature of 80-82° C. for 7-9 min, and then labeled analogically as in the case of PET containers. The final stage of the manufacture process is, for both types of containers, packing into collective packages in multi-packs of 6, 12 or 24 containers and preparing for distribution.

1. The isotonic beverage with chelates containing carbohydrates, mineral amino acid chelates, L-carnitine, sweeteners, flavouring substances, and a diluent wherein the beverage contains up to 0.1% wt of mineral components in the form of mineral amino acid chelates, preferably bisglycinates and glycinates, from 0.1 to 50 wt % of isomaltulose, up to 15 wt % of L-carnitine and/or its derivatives, from 5 to 84% of carbohydrates, up to 50 wt % of bioactive substances, up to 50 wt % of flavouring substances, up to 80 wt % of polyalcohols, diluents, tableting aids, stabilizers, antioxidants, dyes, and 0.013-25 wt % of sweeteners, whereby its osmolarity is 275-295 mOsm/kg and pH is 2-5.

2. Beverage from claim 1, wherein as the mineral components it contains magnesium and/or calcium and/or potassium glycinate in the form of bisglycinates

3. A beverage from claim 1 or 2 wherein as mineral components in the form of bisglycinates the beverage contains magnesium and/or calcium and/or potassium glycinate, and preferably contains also glycinates of zinc, iron, manganese, molybdenum, chromium, copper and/or selenium.

4. Beverage from claim 1, wherein as mineral components it preferably contains other mineral components in organic and/or inorganic form.

5. Beverage from claim 1 or 4 wherein as mineral components it contains chelates of amino acids other than glycine, preferably L-arginine, L-methionine, L-lysine, and L-asparagine acid.

6. Beverage from claim 1 wherein as carbohydrates it contains glucose and/or fructose and/or sucrose and/or isomaltulose.

7. Beverage from claim 1 wherein as bioactive substances it contains magnesium creatine chelate, oligopeptides, preferably glutamine peptide, proteins, preferably whey protein isolate, caffeine, salt, taurine, theanine, unsaturated fatty acids, preferably omega 3-6-9 fatty acids, astaxanthin, ubiquinone, preferably coenzyme Q10, hydroxymethyl butyrate (HMB), vegetable and fruit extracts, preferably green tea extract, ginseng extract, citrus fruit extract or other active substances, preferably chemical compounds showing physiological action.

8. Beverage from claim 1 wherein as flavouring substances it contains synthetic, synthetic-natural, or natural substances in the form of a liquid, powder, emulsion, or seasoning, preferably grapefruit juice concentrate or cherry juice concentrate, orange juice concentrate, orange flavour, pineapple flavour, apple flavour, lemon flavour, pineapple-strawberry seasoning and forest fruit flavour, preferably raspberry-blackberry flavor.

9. Beverage from claim 1 wherein as stabilizers it preferably contains gum Arabic, guar gum, carrageen gum, cellulose gum, xanthan gum, and glycerol and vegetable resin esters.

10. Beverage from claim 1 wherein as antioxidants it preferably contains citric acid or sodium citrate or calcium lactate or ascorbic acid or calcium ascorbate.

11. Beverage from claim 1 wherein as dyes it preferably contains brilliant green and/or quinoline yellow or Allura red or brilliant blue or ammonia caramel.

12. Beverage from claim 1 wherein as preservatives it contains sodium benzoate and/or potassium sorbate.

13. Beverage from claim 1 wherein as sweeteners it contains liquid glucose and crystalline sugar or sugar powder, or aspartame and/or acesulphame K and/or cyclamic acid and salts of this acid and/or saccharine or its salts and/or sucralose.

14. Beverage from claim 1 wherein as polyalcohols it preferably contains sorbitol, mannitol, xylitol, and erithritol.

15. Beverage from claim 1 wherein as diluents it preferably contains fructose, sorbitol, mannitol, xylitol, and erithritol.

16. Beverage from claim 1 wherein as anti-caking substances it preferably contains magnesium stearate and/or colloidal silica.

17. Beverage from claim 1 wherein as vitamins it preferably contains vitamin premix composed of Vitamin A, C, B₁, B₂, B₆, B₁₂, and H, panthotenic acid, Vitamin D and E, niacin, and folic acid.

18. Beverage from claims 1-17 may take a form of an instant powder for dissolving in water.

19. Beverage from claims 1-17 may be a liquid solution.
